(19) United States
(12) Patent Application Publication
(10) Pub. No.: US 2025/0357008 A1
(43) Pub. Date: Nov. 20, 2025

(54) SYSTEMS AND METHODS FOR FALL PREVENTION

(71) Applicant: Prev.Ai LLC, Raleigh, NC (US)

(72) Inventors: Jonathan Hills, Brooklyn, NY (US); James Taylor, Arden, NC (US)

(73) Assignee: Prev.Ai LLC, Raleigh, NC (US)

(21) Appl. No.: 19/202,745

(22) Filed: May 8, 2025

Related U.S. Application Data
(60) Provisional application No. 63/647,165, filed on May 14, 2024.

(51) Int. Cl. G16H 50/30; G16H 10/20; G16H 10/60; G16H 15/00

(52) U.S. Cl. CPC G16H 50/30; G16H 10/20; G16H 10/60; G16H 15/00

(57) ABSTRACT

A virtual system of fall prevention and home safety and optionally hospital discharge, comprising an application to implement a method comprising obtaining data on a resident of the resident's home environment and obtaining data on the resident's health;
analyzing said data with an algorithm and generating a Personalized Fall Risk Index (PFRI) and an Environmental Fall Risk Index (EFRI), thereby allowing a clinician and/or an artificial intelligence (“AI”) system to assess the resident's fall risk at the home environment and to generate a list of personalized recommendations;
suggesting products to said user and/or said resident, connecting said user and/or resident to local contractors, installers and other professionals, and/or connect the user and/or resident to source of grant funding. The PFRI is generated through applied coefficients to predictor variables. The EFRI is derived first as unbound score by averaging three individual scores: weighted average response score, room score, and occupancy score.

### TECHNICAL FIELD

This disclosure relates to fall prevention, home safety, hospital discharge planning, and home safety modification.

### BACKGROUND

Falling at home is a real concern, for anyone but especially for the elderly, the disabled, babies, etc.

Many elderly break hip after a fall, requiring hospitalization and may result in death or debilitation. Risk of falling of course is not limited to the elderly or someone who lives alone.

The elderly especially, and certainly true for others, upon discharge from the hospital, is at high risk for falling at home.

This is especially critical during transitions of care-such as after a hospital discharge, a rehabilitation stay, or surgical procedure-when individuals are at heightened risk of falls due to changes in mobility, medication, or home environment challenges. Hospitals and health systems are increasingly responsible for ensuring safe discharges and minimizing avoidable readmissions, yet many lack tools that assess the home setting with clinical precision. An integrated approach to fall prevention that connects health status to environmental safety is needed.

### SUMMARY

In one aspect, this disclosure provides a virtual system of fall prevention and home safety and optionally hospital discharge, comprising an application (an “app”) to implement a method of:
obtaining data on a resident in a home in need of fall prevention, home safety, and optionally hospital discharge, by uploading videos and/or photos of the resident's home environment and obtaining data on the resident's health;
analyzing said data with an algorithm and generating a Personalized Fall Risk Index (PFRI) and an Environmental Fall Risk Index (EFRI), each of which is unique to said resident, thereby allowing a clinician and/or an artificial intelligence (“AI”) system to assess the resident's fall risk at the home environment and to generate a list of personalized recommendations for said resident and for a user of the method and system covering areas of concern in each room of the resident's home environment;
based on said recommendations, suggesting products to said user and/or said resident, connecting said user and/or resident to local contractors, installers and other professionals, who can address the user's and/or resident's needs in the resident's home environment, and/or connect the user and/or resident to source of grant funding;
wherein the PFRI is generated through applied coefficients to predictor variables, and is a representation of the resident's relative fall risk;
wherein the EFRI is derived first as unbound score by averaging three individual scores: weighted average response score, room score (inherent risk), and occupancy score (time spent in room); wherein the unbounded score is adjusted within a clinician's provided score range for a comprehensive room-level fall risk assessment.

This disclosure also provides a method of fall prevention and home safety and optionally hospital discharge comprising:
obtaining data on a resident in a home in need of fall prevention, home safety, and optionally hospital discharge, by uploading videos and/or photos of the resident's home environment and obtaining data on the resident's health;
analyzing said data with an algorithm and generating a Personalized Fall Risk Index (PFRI) and an Environmental Fall Risk Index (EFRI), each of which is unique to said resident, thereby allowing a clinician and/or an artificial intelligence (“AI”) system to assess the resident's fall risk at the home environment and to generate a list of personalized recommendations for said resident and for a user of the method and system covering areas of concern in each room of the resident's home environment;
based on said recommendations, suggesting products to said user and/or said resident, connecting said user and/or resident to local contractors, installers and other professionals, who can address the user's and/or resident's needs in the resident's home environment, and/or connect the user and/or resident to source of grant funding;
wherein the PFRI is generated through applied coefficients to predictor variables, and is a representation of the resident's relative fall risk;
wherein the EFRI is derived first as unbound score by averaging three individual scores: weighted average response score, room score (inherent risk), and occupancy score (time spent in room); wherein the unbounded score is adjusted within a clinician's provided score range for a comprehensive room-level fall risk assessment.

Numerous other aspects are provided in accordance with these and other aspects of the invention. Other features and aspects of the present invention will become more fully apparent from the following detailed description and the appended claims.

### DETAILED DESCRIPTION

As used herein, the word “a” or “plurality” before a noun represents one or more of the particular noun.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

All ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10” or “5 to 10” or “5-10” should be considered to include the end points 5 and 10.

It is further to be understood that the feature or features of one embodiment may generally be applied to other embodiments, even though not specifically described or illustrated in such other embodiments, unless expressly prohibited by this disclosure or the nature of the relevant embodiments. Likewise, compositions and methods described herein can include any combination of features and/or steps described herein not inconsistent with the objectives of the present disclosure. Numerous modifications and/or adaptations of the compositions and methods described herein will be readily apparent to those skilled in the art without departing from the present subject matter.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, controls.

The disclosed systems and methods address critical gaps in transitional care by equipping healthcare providers, discharge planners, and home-based care teams with a scalable method to assess and reduce fall risk and improve home accessibility. This includes preoperative and post-discharge planning, supporting safer recoveries and reducing the likelihood of readmissions. By linking health and home data, the system ensures recommendations are clinically relevant and personalized to the individual's medical needs and recovery context.

In one aspect, this disclosure provides a virtual system of fall prevention and home safety. The system is optionally of hospital discharge. The system comprises an application (an “app”), (in some embodiments, the app is an Internet web-based app) to implement the following method. The method comprises:
obtaining data on a resident in a home in need of fall prevention, home safety, and optionally hospital discharge, by uploading videos and/or photos of the resident's home environment and obtaining data on the resident's health;
analyzing said data with an algorithm and generating a Personalized Fall Risk Index (PFRI) and an Environmental Fall Risk Index (EFRI), each of which is unique to said resident, thereby allowing a clinician and/or an artificial intelligence (“AI”) system to assess the resident's fall risk at the home environment and to generate a list of personalized recommendations for said resident and for a user of the method and system covering areas of concern in each room of the resident's home environment;
based on said recommendations, suggesting products to said user and/or said resident, connecting said user and/or resident to local contractors, installers and other professionals, who can address the user's and/or resident's needs in the resident's home environment, and/or connect the user and/or resident to source of grant funding;
wherein the PFRI is generated through applied coefficients to predictor variables, and is a representation of the resident's relative fall risk;
wherein the EFRI is derived first as unbound score by averaging three individual scores: weighted average response score, room score (inherent risk), and occupancy score (time spent in room); wherein the unbounded score is adjusted within a clinician's provided score range for a comprehensive room-level fall risk assessment.

In some embodiments, the resident is to be discharged from a hospital. In other embodiments, the resident is not to be discharged from a hospital. In some embodiments, the method further comprising delivering an interactive virtual home safety and fall prevention report to the user and/or resident.

In another aspect, a method is provided of fall prevention and home safety and optionally hospital discharge comprising:

obtaining data on a resident in a home in need of fall prevention, home safety, and optionally hospital discharge, by uploading videos and/or photos of the resident's home environment and obtaining data on the resident's health;
analyzing said data with an algorithm and generating a Personalized Fall Risk Index (PFRI) and an Environmental Fall Risk Index (EFRI), each of which is unique to said resident, thereby allowing a clinician and/or an artificial intelligence (“AI”) system to assess the resident's fall risk at the home environment and to generate a list of personalized recommendations for said resident and for a user of the method and system covering areas of concern in each room of the resident's home environment;
based on said recommendations, suggesting products to said user and/or said resident, connecting said user and/or resident to local contractors, installers and other professionals, who can address the user's and/or resident's needs in the resident's home environment, and/or connect the user and/or resident to source of grant funding;
wherein the PFRI is generated through applied coefficients to predictor variables, and is a representation of the resident's relative fall risk;
wherein the EFRI is derived first as unbound score by averaging three individual scores: weighted average response score, room score (inherent risk), and occupancy score (time spent in room); wherein the unbounded score is adjusted within a clinician's provided score range for a comprehensive room-level fall risk assessment.

In some embodiments, the resident is to be discharged from a hospital. In other embodiments, the resident is not to be discharged from a hospital. In some embodiments, the method further comprising delivering an interactive virtual home safety and fall prevention report to the user and/or resident.

The term “resident” is a person residing in a home. This person is in need of fall prevention and home safety. In some embodiments, the resident is elderly. In some embodiments, the resident lives alone. In other embodiments, the resident is disabled. In some embodiments, the resident has autism. In some embodiments, the resident is a baby/toddler. In some embodiments, the resident has chronic disease.

The term “user” referred herein can be any person or persons, and can be an organization. The user does not need to be the resident. The user is anyone in need of fall prevention, home safety, or about to be discharged from the hospital (thus, the user can be a resident), or a caregiver(s), caregiver organization, relative(s), of that person. In some embodiments, the user is elderly. In some embodiments, the user lives alone. In other embodiments, the user is disabled. In some embodiments, the user has autism. In some embodiments, the user is a baby/toddler or a baby/toddler's parent(s), with the baby/toddler residing at the home. In some embodiments, the user has chronic disease. The user can be a social service worker(s) or an organization with social service worker(s). The user can be relative(s) of the person who resides in the home. The user can be a home health aide of the person residing at the home. The person residing in the home can be the user as well.

The recommendations provided to the user can also be provided to the user's healthcare professional(s).

The videos and/or photos of the home environment can be obtained by any methods and by using any video and/or photo capturing machine. The videos and/or photos capture details of each room of the resident's home environment. The more details the better. The videos and/or photos can be updated as a room changes, which results in updating the disclosed system and the disclosed method. However, a room has certain inherent risks that do not change, such as location of stairs, etc. The health-related survey is a comprehensive survey of the resident's health history. The survey answers can also be provided, at least in part, by the user's clinician. The survey can be done on paper, on a word processor, etc. Data on the resident's health can be provided by other means, such as by the resident's electronic health record.

In some embodiments, health-related data are obtained from electronic health records (EHRs), patient portals, or health information systems, subject to appropriate authorization. These data enhance the accuracy of the Personalized Fall Risk Index by reflecting current medications, clinical diagnoses, recent procedures, or mobility assessments recorded by care teams. Integration with health records allows the system to align more closely with existing healthcare workflows and discharge planning protocols.

While the system/method are primarily designed to support healthcare professionals, families, and caregivers in fall prevention and home safety planning, recommendations may also be developed through direct clinical review of the resident's health and home environment, independent of the risk scores generated by the system, allowing for flexibility in use cases where algorithmic scoring may not be necessary or preferred. The recommendations generated by the system may include environmental modifications (e.g., grab bars, ramps), smart home technologies (e.g., fall detection sensors), durable and home medical equipment (e.g., hospital beds, shower chairs), and discharge support resources. In some embodiments, the recommendations may also encompass physical therapy (PT), exercise regimens, or other wellness-focused guidance tailored to the individual's health profile and functional capacity.

In some embodiments, the user may be a fiduciary, legal guardian, or trust officer responsible for managing the living arrangements and safety of a beneficiary, such as in the case of special needs trusts, elder care, or other legally supervised housing. The system may assist such users in evaluating and documenting fall risk and safety needs to fulfill their duty of care.

In other embodiments, the system may be used by insurance carriers, case managers, or employers involved in coordinating return-to-home transitions following workplace injury or disability-related hospitalization. The method supports proactive identification of environmental risks that could impede recovery or lead to reinjury.

In further embodiments, the user may be a family member or household decision-maker seeking to proactively identify safety improvements in the home—for example, to childproof a residence for an infant or toddler, or to prepare a home for aging in place. The system enables such users to receive personalized room-by-room guidance without clinical intervention.

In some embodiments, the system may be deployed by healthcare institutions or public agencies, such as skilled nursing facility discharge teams, Medicaid programs, or nonprofit housing organizations, to assess home readiness, allocate resources, or document intervention needs in support of continued safe residence.

The algorithm (software) used is in a computer-readable medium and is to be read by a computer, and is used to analyze the data obtained on the home environment of the resident and the health data on the resident and to then generate the PFRI and EFRI of the resident's home environment. Any suitable algorithm can be used. A person of ordinary skill in the art can write such an algorithm.

The PFRI is a representation of an individual's relative fall risk, integrating demographic and medical-history responses using a logistic function. The composite score is determined through applied coefficients to predictor variables. Confidence intervals are considered, providing upper and lower probability ranges. The PFRI facilitates clinicians in assessing a resident's home environment and making individualized recommendations based on the person's unique health profile. The components of the PFRI also automatically link educational content into a user's report, enhancing understanding and application of recommendations. The PFRI was developed by considering more than 400,000 data points on fall risk. Odds ratios for each question are grouped by category, listed, and simulated user responses calculate the composite score. Output probability is categorized based on a defined mapping, adjustable as needed.

The EFRI is calculated in two stages, deriving an unbounded score by averaging three individual scores: the weighted average response score, room score (inherent risk), and occupancy score (time spent in the room). The unbounded score is then adjusted within the clinician's provided score range for a comprehensive room-level fall risk assessment. This ensures the novel EFRI is always anchored to a clinician's assessment and represents a true blend of both scores derived from algorithms and clinical experience, training and acumen. An overall composite score for the entire home is calculated based on the weighted average of each room's composite score. The EFRI also provides an individual score for each assessed room, allowing users to allocate resources to areas of highest concern.

In the disclosed method and system, user inputs and clinician-provided scores, along with question weights are used to calculate the composite score for each room. The composite score for each room is thus based on user responses, room factor, and occupancy factor. The final home score reflects overall fall risk, considering both the user's home environment and health profile.

The disclosed system and method allow a clinician to assess the user's fall risk at the home environment and to generate a list of personalized recommendations for the user covering areas of concern in each room of the user's home environment. And based on these recommendations, the system and method suggest product links to the user and/or connect the user to local contractors, installers, and other professionals who can best address the user's needs in the user's home environment.

A source of grant funding is funding that the person residing at the home can qualify for, but may be not aware of. The funding may be from a government or private source.

The term “clinician” is used interchangeably with the term “physician” and the term “doctor.”

The term “app” is a self-contained software package that allows users to perform specific tasks on a mobile or desktop device.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the appended claims. Thus, while only certain features of the invention have been illustrated and described, many modifications and changes will occur to those skilled in the art. It is therefore to be understood that the appended claims are intended to cover all such modifications and changes as fall within the true spirit of the invention.

What is claimed is:

1. A virtual system of fall prevention and home safety and optionally hospital discharge, comprising an application (“app”) to implement a method of:
obtaining data on a resident in need of fall prevention, home safety, and optionally hospital discharge, by uploading media, including video, photos, or other visual data, of the user's home environment and providing answers from the user to a health-related survey;
analyzing said data with an algorithm and generating a Personalized Fall Risk Index (PFRI) and an Environmental Fall Risk Index (EFRI), each of which is unique to said resident, thereby allowing a clinician and/or an artificial intelligence (“AI”) system to assess the resident's fall risk at the home environment and to generate a list of personalized recommendations for said resident and for a user of the method and system covering areas of concern in each room of the resident's home environment;
based on said recommendations, suggesting products to said user and/or said resident, connecting said user and/or resident to local contractors, installers and other professionals, who can address the user's and/or resident's needs in the resident's home environment, and/or connect the user and/or resident to source of grant funding;
wherein the PFRI is generated through applied coefficients to predictor variables, and is a representation of the resident's relative fall risk;
wherein the EFRI is derived first as unbound score by averaging three individual scores:
weighted average response score, room score (inherent risk), and occupancy score (time spent in room); wherein the unbounded score is adjusted within a clinician's provided score range for a comprehensive room-level fall risk assessment.

2. The system of claim 1, wherein the user is to be discharged from a hospital.

3. The system of claim 1, wherein the user is not to be discharged from a hospital.

4. The system of any of claim 1, wherein the app is a web-based app.

5. The system of any of claim 1, wherein said method further comprising delivering an interactive virtual home safety and fall prevention report to the user.

6. A method of fall prevention and home safety and optionally hospital discharge comprising:
obtaining data on a resident in a home in need of fall prevention, home safety, and optionally hospital discharge, by uploading media, including video, photos, or other visual data, of the resident's home environment and obtaining data on the resident's health;
analyzing said data with an algorithm and generating a Personalized Fall Risk Index (PFRI) and an Environmental Fall Risk Index (EFRI), each of which is unique to said resident, thereby allowing a clinician and/or an artificial intelligence (“AI”) system to assess the resident's fall risk at the home environment and to generate a list of personalized recommendations for said resident and for a user of the method and system covering areas of concern in each room of the resident's home environment;
based on said recommendations, suggesting products to said user and/or said resident, connecting said user and/or resident to local contractors, installers and other professionals, who can address the user's and/or resident's needs in the resident's home environment, and/or connect the user and/or resident to source of grant funding;
wherein the PFRI is generated through applied coefficients to predictor variables, and is a representation of the resident's relative fall risk;
wherein the EFRI is derived first as unbound score by averaging three individual scores:
weighted average response score, room score (inherent risk), and occupancy score (time spent in room); wherein the unbounded score is adjusted within a clinician's provided score range for a comprehensive room-level fall risk assessment.

7. The method of claim 6, wherein the resident is to be discharged from a hospital.

8. The method of claim 6, wherein the resident is not to be discharged from a hospital.

9. The method of any of claim 6, herein the app is a web-based app.

10. The method of any of claim 6, further comprising delivering an interactive virtual home safety and fall prevention report to the user.
